(19) United States
(12) Patent Application Publication
(10) Pub. No.: US 2021/0113813 A1
(43) Pub. Date: Apr. 22, 2021

(54) Steerable Neurovascular Guide Catheter

(71) Applicant: ENZIAN NEUROSURGICAL, LLC, Santa Barbara, CA (US)

(72) Inventors: Alois Zauner, Santa Barbara, CA (US); Stephen Laguette, Santa Barbara, CA (US)

(21) Appl. No.: 17/071,165

(22) Filed: Oct. 15, 2020

Related U.S. Application Data
(60) Provisional application No. 62/916,495, filed on Oct. 17, 2019.

(51) Int. Cl. A61M 25/01; A61M 25/00

(52) U.S. Cl. CPC A61M 25/0133; A61M 2025/09125; A61M 25/0068; A61M 25/0026

(57) ABSTRACT

An Improved Steerable Neurovascular Guide Catheter with torquable means for distal tip deflection and lockable tip deflections to adjust catheter tip deflector and shape improves access and delivery of therapies by one hand of a user.

### CROSS REFERENCE TO RELATED APPLICATIONS

This application claims full priority benefit from U.S. Ser. No. 62/916,495, filed Oct. 17, 2019, the entirety of which is expressly incorporated by reference.

### BACKGROUND OF THE DISCLOSURES

Many guide catheters are known, and all patents referenced in web-sites, on products and labels which are publicly available as associated with CORDIS® (Envoy®), MEDTRONIC® (Navien®); MICROVENTION/TERUMO® (Chaperon®); Penumbra® (Neuron/Benchmark®); Bait USA® (BALLAST 88®) and/or Stryker® (DAC®); Asahi® (Fubuki®) or any other brands referenced herein are expressly incorporated by reference as if fully set forth herein, each having been reviewed and compare to the novelties added herein and distinguished, it is respectfully proposed. Likewise, all proprietary marks and other trade indicia remain property of their respective owners, and are merely used to define the state-of-the-art.

### OBJECTS AND SUMMARY OF THE INVENTION

The following separate improved aspects are disclosed herein and part and parcel of the present inventions, according to embodiments that are shown and taught illustrated below, described and claimed, for an improved steerable neurovascular guide catheter.

According to embodiments, there are provided improved catheters combining pre-formed shaped tips with multi-lumen bodies having integral wire guides and unique 3-D shapes.

According to embodiments, there is provided a system for neurovascular guidance with at least one of stainless steel, braided and Nitinol® types of devices, over-the-wire, which likewise injects contrast and has a low profile customized ergonomic luer manifold providing one-handled access and delivery of various therapies.

### BRIEF DESCRIPTION OF THE DRAWINGS

Various preferred embodiments are described herein with references to the drawings in which merely illustrative views are offered for consideration, whereby:

FIG. 1 is a dispositive and schematic view, showing aspects of the IMPROVED STEERABLE NEUROVASCULAR GUIDE CATHETER of the present invention;

FIG. 2 is a dispositive and schematic view, showing aspects of the IMPROVED STEERABLE NEUROVASCULAR GUIDE CATHETER of the present invention;

FIG. 3 is a dispositive and schematic view, showing aspects of the IMPROVED STEERABLE NEUROVASCULAR GUIDE CATHETER of the present invention;

FIG. 4 is a dispositive and schematic view, showing aspects of the IMPROVED STEERABLE NEUROVASCULAR GUIDE CATHETER of the present invention;

FIG. 5 is a dispositive and schematic view, showing aspects of the IMPROVED STEERABLE NEUROVASCULAR GUIDE CATHETER of the present invention;

FIG. 6 is a side view, dispositive and schematic view, showing aspects of the IMPROVED STEERABLE NEUROVASCULAR GUIDE CATHETER of the present invention;

FIG. 7 is also a side plan view, dispositive and schematic view, showing aspects of the IMPROVED STEERABLE NEUROVASCULAR GUIDE CATHETER of the present invention;

FIG. 8 is a dispositive and schematic view, showing aspects of the IMPROVED STEERABLE NEUROVASCULAR GUIDE CATHETER of the present invention;

FIG. 9 is a dispositive and schematic view, showing aspects of the IMPROVED STEERABLE NEUROVASCULAR GUIDE CATHETER of the present invention;

FIG. 10 is a dispositive and schematic view, showing aspects of the IMPROVED STEERABLE NEUROVASCULAR GUIDE CATHETER of the present invention;

FIG. 11 is a dispositive and schematic view, showing aspects of the IMPROVED STEERABLE NEUROVASCULAR GUIDE CATHETER of the present invention;

FIG. 12 is a dispositive and schematic view, showing aspects of the IMPROVED STEERABLE NEUROVASCULAR GUIDE CATHETER of the present invention; and,

FIG. 13 is another schematic showing radial access modifications inter alia.

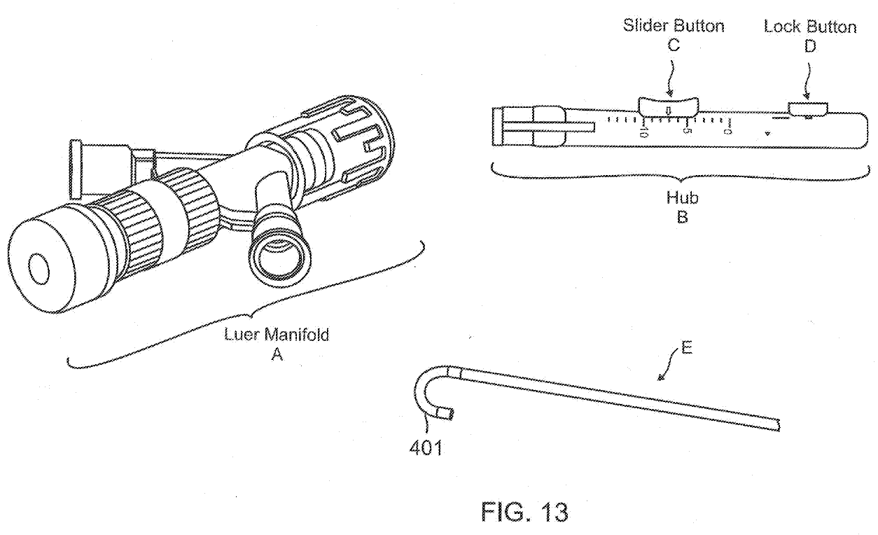

Corresponding written descriptions at this time are in the place of reference characters to indicate corresponding components throughout the several views of the drawings, this is interchanged with several reference designating sets of numbers to show the invention. Skilled artisans will appreciate that elements in the figures are illustrated for simplicity and clarity, and have not necessarily been drawn to scale. For example, the dimensions of some of the elements in the figures may be exaggerated relative to other elements to help to improve understanding of various embodiments of the present invention. Also, common but well-understood elements that are useful or necessary in a commercially feasible embodiment are often not depicted in order to facilitate a less obstructed view of these various embodiments of the present invention.

### DETAILED DESCRIPTIONS OF THE INVENTION

The present inventor has improved and thus reinvented a novel medical device which addresses, as disclosed, clinical performance needs to provide access to and be a conduit for neurovascular devices, while injecting contrast. The prior art was investigated and studied, showing numerous areas to be improved as offered for consideration as aspects of the present invention. Namely, the hand and performance aspects required to overcome narrow and tortuous vessels has been optimized and dictates the scope of improvement of the instant system, and present inventions. By way of state-of-the-art example, the top eight catheters used were profiled including but not limited to the following, along with type of therapy used.

| | | |
|---|---|---|
| Cordis | Envoy | Stainless steel braid |
| Medtronic | Navien | Braided/coil |
| Microvention | Chaperon | Braided |
| Mivi | Mivi Q | Braided |
| Penumbra | Neuron/Benchmark | Braided/coil |
| Stryker | DAC | Braided |
| Asahi | Fubuki | Stainless steel braid |
| Balt-USA | Ballast 88 | Braided |

Steerable Neurovascular Guide Catheter: What is new, novel and non-obvious, it is respectfully proposed, and subject to US Letters Patent treatment and is offered for consideration is a system focused upon an improved Steerable Neurovascular Guide Catheter

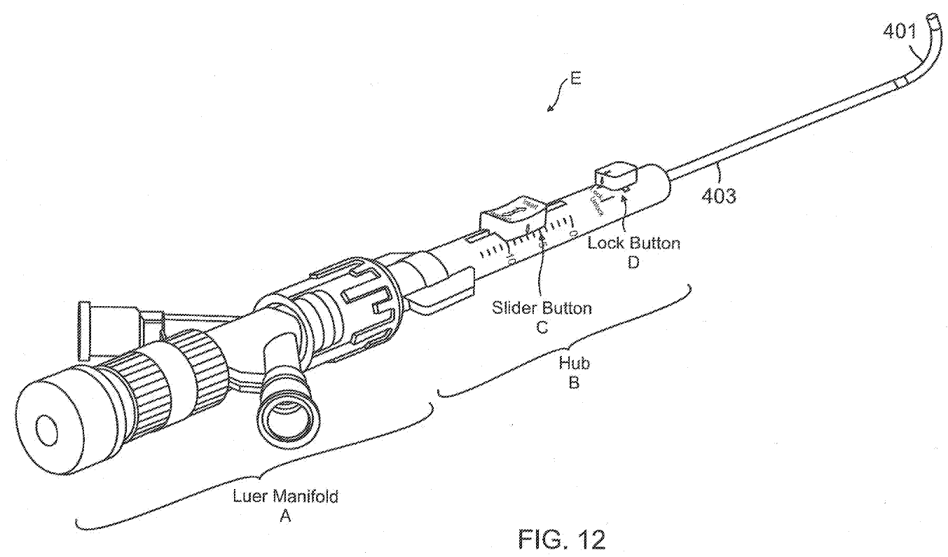

Design Requirements: Improved distal navigation, ergonomics and ease of use are each desiderata. Doing so specfically via improved proximal and distal stability. The structural flexibility and modular nature of the system supports the evolution and development of slightly different pre-formed shapes, sizing's and tip compliance changes—for example to enable future neurovascular radial approaches. See, for example FIG. 12.

Key Design Features of illustrated embodiments; integral wire guide with deflectable distal tip 401 having a plurality of pre-set forms/shapes, variable and lockable tip deflection, co-extruded guide catheter tubing with non-circular shape and optimized material properties, extra soft molded distal tip 401 for non-traumatic use, and a customized ergonomic luer manifold provides ease of use.

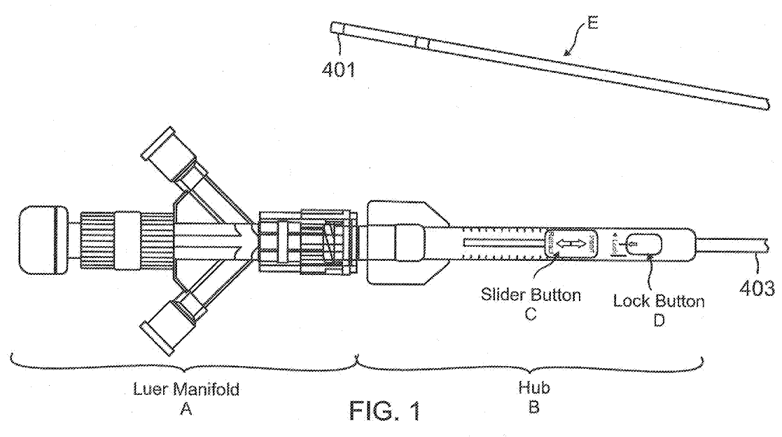

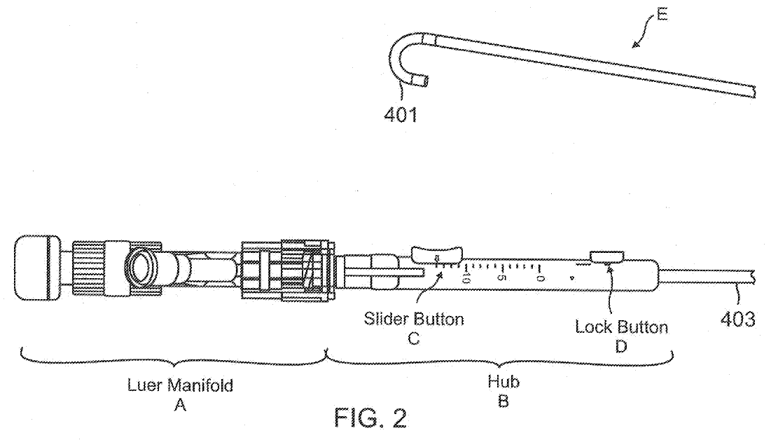

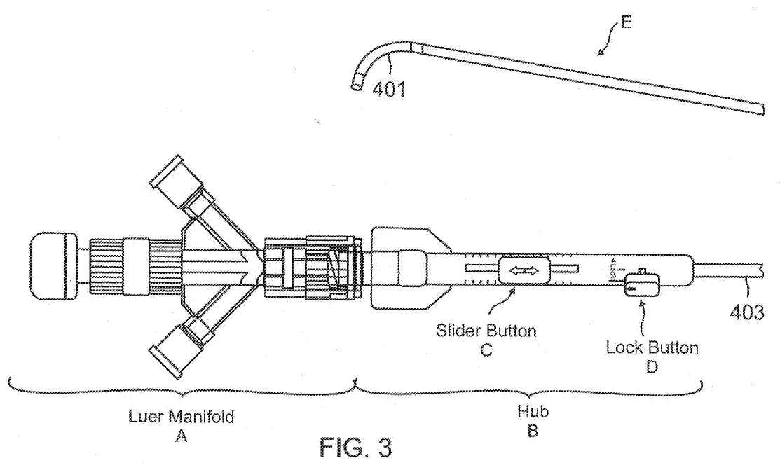

Example One: Integral guide wire with deflectable distal tip 401: Referring now to FIG. 1, FIG. 2 and FIG. 3, specifically, “Steerable”, includes distal tip 401.

Variable and lockable tip deflection, is shown by catheter side mount 403.

Co-extruded guide catheter tubing with non-circular shape and optimized material properties. Likewise, shown in the figures, low-profile OD extruded.

Improved Deflection. FIG. 1 shows the example device, which has wire guide fully inserted, making the distal tip 401, straightened, with lock button D radially locked. FIG. 2 shows a second position for the distal tip 401 with wire guide fully retracted and respective buttons C & D and pre-formed, fully hooked configuration of catheter segment E. FIG. 3 shows a third position with wire guide half inserted along with each respective button position, C & D.

The present inventions also feature an extra soft molded distal tip 401 for non-traumatic use, improved distal navigation, proximal and distal stability, in various pre-formed shapes.

Customized ergonomic luer manifold A for ease of use enhances variable flexibility, is modular and allows tortuous vessels to be managed—along with lower chance of air embolization.

Proximal round molding enables the torquable tip to navigate without being impeded by the sheath, in terms of stearic hindrance.

These improvements are part of new product lines for diagnostic and neurointerventional procedures, both the traditional femoral and newer radial approaches are indicated according to the teachings of the present invention. Artisans are readily familiar with changes used to effect radial approaches

According to the current invention, the extra soft tip, is both torquably lockable and steerable at 80 to 115 cm E, as shown in FIG. 1-3 which show the example device with a wire guide fully inserted, making the distal tip 401 straightened, with button D radially locked, and a second position, and third position.

FIG. 2 shows a second position for the distal tip 401 with wire guide fully retracted and respective buttons and fully hooked configuration, or any of the preferred shape.

FIG. 3 shows a third position with wire guide half inserted along with each respective button position, C & D again any pre-formed shape can be made.

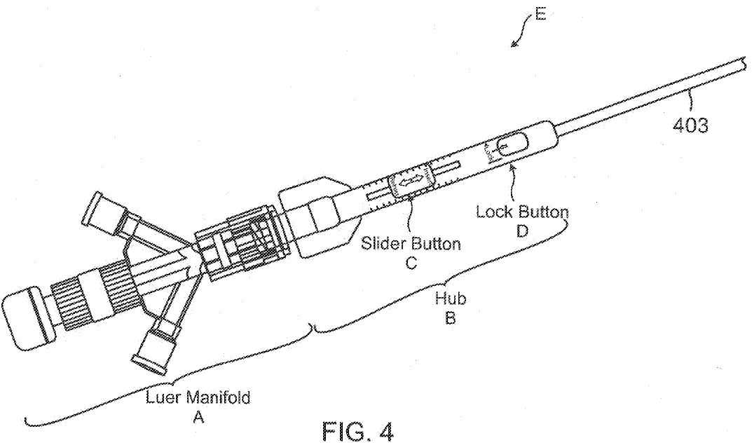

FIG. 4 shows a top view with the novelly shaped device designed to deliver at least a soft pre-formed shaped distal tip 401 having an integral wire guide.

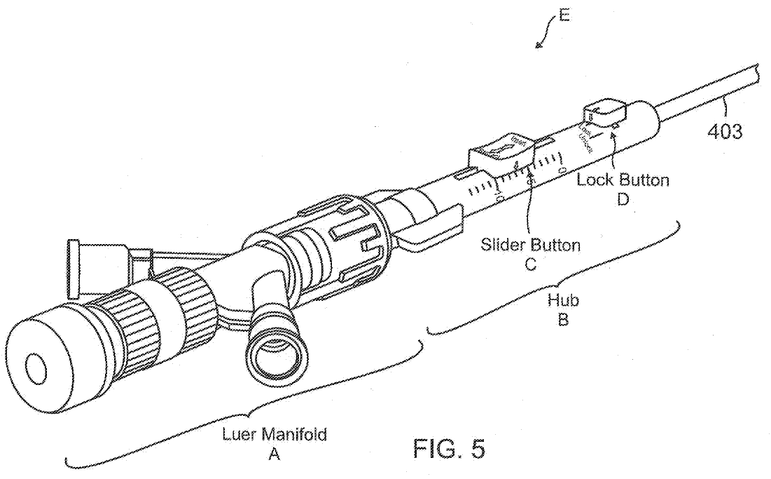

FIG. 5 shows the customized ergonomic luer manifold A from a partial perspective view, along with Hub B which is operable by one hand of a user, owing to its modularity and flexibility, it can be steered into places the prior art cannot, using slider button C, and lock button D.

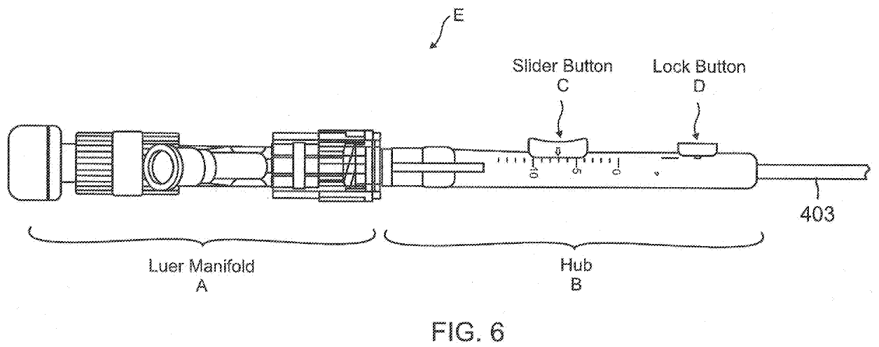

FIG. 6 is a side view showing a low profile easily navigable tool E as described.

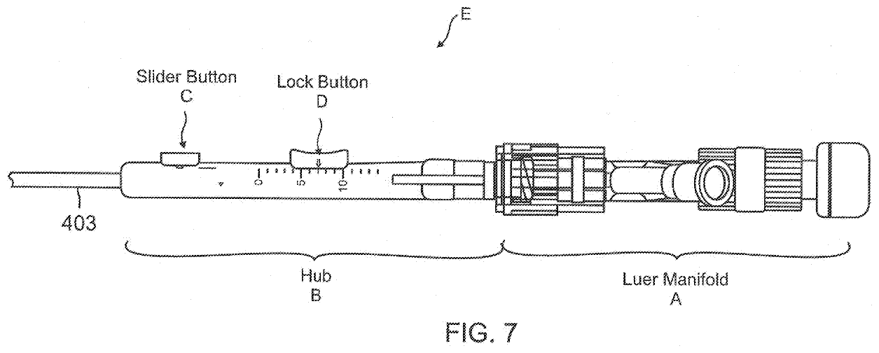

FIG. 7 is also a side plan view, showing the novel planar yet smooth and compact shape of luer manifold A allowing it to slide facilely into tortuous anatomy.

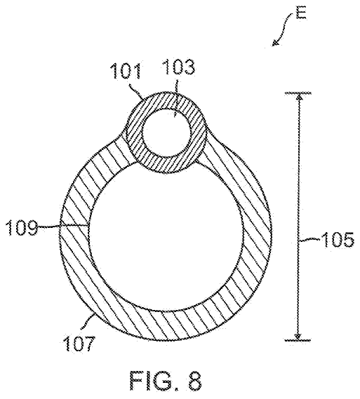

FIG. 8 is a cartoon/schematic not depicted to scale of an embodiment showing illustrative but not limiting size range for the instant teachings. Showing an example guide catheter 403 with 0.006″ wall thickness 101, and 0.018″ ID 103, with 7 Fr (0.092″) height 105 of (0.079″) 6 Fr and ID 109 of 0.058″.

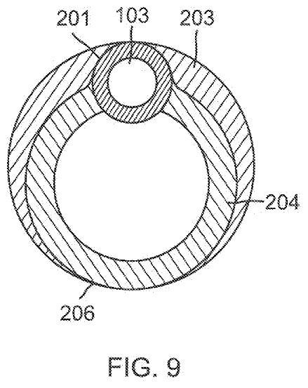

FIG. 9 shows proximal and cross-section with overmolding. Again purely by example the Inner Diameter 103 is 0.018″ wherein the 7 Fr (0.092″) diameter at the top 203 tapers to an OD of 6 Fr (0.079″) at 206.

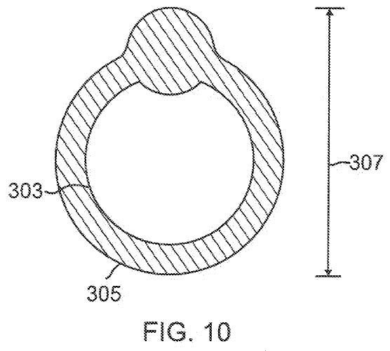

FIG. 10 shows the distal tip 401 cross section of an example device 403, namely Inner Diameter 303 of 0.058″ with an Outer Diameter 305 of 6 Fr (0.079″ cm) and an overall diameter of at least about 7 Fr (0.092″) 307. No limitations as to size are implied by this illustrative example.

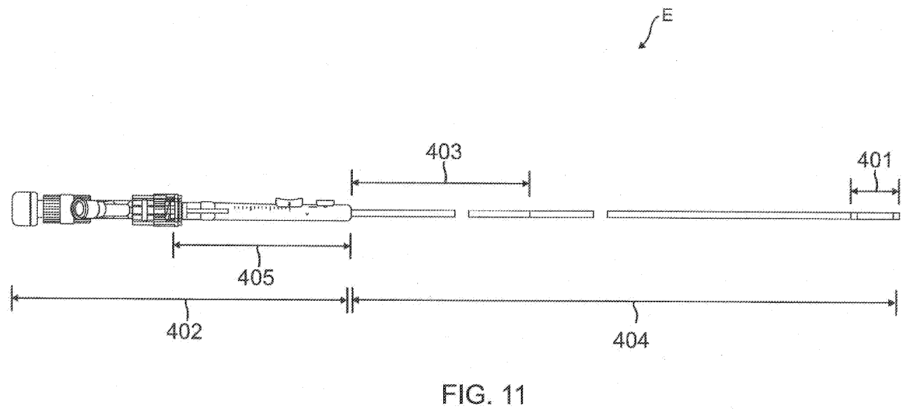

FIG. 11 likewise shows potential sizing for an exemplary embodiment E measuring 14.5 cm, for example for distal tip 401, 12.2 cm for 402 ergonomic assembly of luer manifold (A) with hub (B) length, 50 cm for 403, 115 cm for 404 and 6.35 cm for 405 and injection of contrast is likewise facilitated by the structure.

FIG. 12 also shows a system currently useful for femoral applications and radial with slight modification, namely the change in length, (if coating to hub B) placement of guide catheter only with 403, & hydrophic coating until hub, Optimized OD over time shall be used.

While several embodiments of the present disclosure have been described and illustrated herein, those of ordinary skill in the art will readily envision a variety of other means and/or structures for performing the functions and/or obtaining the results and/or one or more of the advantages described herein, and each of such variations and/or modifications is deemed to be within the scope of the present disclosure. More generally, those skilled in the art will readily appreciate that all parameters, dimensions, materials, and configurations described herein are meant to be exemplary and that the actual parameters, dimensions, materials, and/or configurations will depend upon the specific application or applications for which the teachings of the present disclosure is/are used.

Those skilled in the art will recognize, or be able to ascertain using no more than routine experimentation, many equivalents to the specific embodiments of the disclosure described herein. It is, therefore, to be understood that the foregoing embodiments are presented by way of example only and that, within the scope of the appended claims and equivalents thereto, the disclosure may be practiced otherwise than as specifically described and claimed. The present disclosure is directed to each individual feature, system, article, material, kit, and/or method described herein. In addition, any combination of two or more such features, systems, articles, materials, kits, and/or methods, if such features, systems, articles, materials, kits, and/or methods are not mutually inconsistent, is included within the scope of the present disclosure.

All definitions, as defined and used herein, should be understood to control over dictionary definitions, definitions in documents incorporated by reference, and/or ordinary meanings of the defined terms.

The indefinite articles “a” and “an,” as used herein in the specification and in the claims, unless clearly indicated to the contrary, should be understood to mean “at least one.” The phrase “and/or,” as used herein in the specification and in the claims, should be understood to mean “either or both” of the elements so conjoined, i.e., elements that are conjunctively present in some cases and disjunctively present in other cases. Other elements may optionally be present other than the elements specifically identified by the “and/or” clause, whether related or unrelated to those elements specifically identified, unless clearly indicated to the contrary.

Reference throughout this specification to “one embodiment” or “an embodiment” means that a particular feature, structure, or characteristic described in connection with the embodiment is included in at least one embodiment. Thus, appearances of the phrases “in one embodiment” or “in an embodiment” in various places throughout this specification are not necessarily all referring to the same embodiment. Furthermore, the particular features, structures, or characteristics may be combined in any suitable manner in one or more embodiments.

The terms and expressions which have been employed herein are used as terms of description and not of limitation, and there is no intention, in the use of such terms and expressions, of excluding any equivalents of the features shown and described (or portions thereof), and it is recognized that various modifications are possible within the scope of the claims. Accordingly, the claims are intended to cover all such equivalents.

Reference throughout this specification to “one embodiment,” “an embodiment,” or similar language means that a particular feature, structure, or characteristic described in connection with the embodiment is included in at least one embodiment of the present invention. Thus, appearances of the phrases “in one embodiment,” “in an embodiment,” and similar throughout this specification may, but do not necessarily, all refer to the same embodiment.

Furthermore, the described features, structures, or characteristics of the invention may be combined in any suitable manner in one or more embodiments. In the following description, numerous specific details are provided to provide a thorough understanding of embodiments of the invention. One skilled in the relevant art will recognize, however, that the invention may be practiced without one or more of the specific details, or with other methods, components, materials, and so forth. In other instances, well-known structures, materials, and so forth. In other instances, well-known structures, materials, or operations are not shown or described in detail to avoid obscuring aspects of the invention.

The schematic flow chart diagrams included herein are generally set forth as logical flow chart diagrams. As such, the depicted order and labeled steps are indicative of one embodiment of the presented method. Other steps and methods may be conceived that are equivalent in function, logic, or effect to one or more steps, or portions thereof, of the illustrated method. Additionally, the format and symbols employed are provided to explain the logical steps of the method and are understood not to limit the scope of the method. Although various arrow types and line types may be employed in the flow chart diagrams, they are understood not to limit the scope of the corresponding method. Indeed, some arrows or other connectors may be used to indicate only the logical flow of the method. For instance, an arrow may indicate a waiting or monitoring period of unspecified duration between enumerated steps of the depicted method. Additionally, the order in which a particular method occurs may or may not strictly adhere to the order of the corresponding steps shown.

Unless otherwise indicated, all numbers expressing quantities of ingredients, properties such as molecular weight, reaction conditions, and so forth used in the specification and claims are to be understood as being modified in all instances by the term “about.” Accordingly, unless indicated to the contrary, the numerical parameters set forth in the specification and attached claims are approximations that may vary depending upon the desired properties sought to be obtained by the present invention. At the very least, and not as an attempt to limit the application of the doctrine of equivalents to the scope of the claims, each numerical parameter should at least be construed in light of the number of reported significant digits and by applying ordinary rounding techniques. Notwithstanding that the numerical ranges and parameters setting forth the broad scope of the invention are approximations, the numerical values set forth in the specific examples are reported as precisely as possible. Any numerical value, however, inherently contains certain errors necessarily resulting from the standard deviation found in their respective testing measurements.

The terms “a,” “an,” “the” and similar referents used in the context of describing the invention (especially in the context of the following claims) are to be construed to cover both the singular and the plural, unless otherwise indicated herein or clearly contradicted by context. Recitation of ranges of values herein is merely intended to serve as a shorthand method of referring individually to each separate value falling within the range. Unless otherwise indicated herein, each individual value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided herein is intended merely to better illuminate the invention and does not pose a limitation on the scope of the invention otherwise claimed. No language in the specification should be construed as indicating any non-claimed element essential to the practice of the invention.

Groupings of alternative elements or embodiments of the invention disclosed herein are not to be construed as limitations. Each group member may be referred to and claimed individually or in any combination with other members of the group or other elements found herein. It is anticipated that one or more members of a group may be included in, or deleted from, a group for reasons of convenience and/or patentability. When any such inclusion or deletion occurs, the specification is deemed to contain the group as modified thus fulfilling the written description of all Markush groups used in the appended claims.

Certain embodiments of this invention are described herein, including the best mode known to the inventors for carrying out the invention. Of course, variations on these described embodiments will become apparent to those of ordinary skill in the art upon reading the foregoing description. The inventor expects skilled artisans to employ such variations as appropriate, and the inventors intend for the invention to be practiced otherwise than specifically described herein. Accordingly, this invention includes all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context.

Specific embodiments disclosed herein may be further limited in the claims using consisting of or consisting essentially of language. When used in the claims, whether as filed or added per amendment, the transition term “consisting of” excludes any element, step, or ingredient not specified in the claims. The transition term “consisting essentially of” limits the scope of a claim to the specified materials or steps and those that do not materially affect the basic and novel characteristic(s). Embodiments of the invention so claimed are inherently or expressly described and enabled herein.

In closing, it is to be understood that the embodiments of the invention disclosed herein are illustrative of the principles of the present invention. Other modifications that may be employed are within the scope of the invention. Thus, by way of example, but not of limitation, alternative configurations of the present invention may be utilized in accordance with the teachings herein. Accordingly, the present invention is not limited to that precisely as shown and described.

What is claimed is:

1. An improved steerable neurovascular guide catheter, which comprises, in combination:
at least a soft pre-formed shaped distal tip straightened with an internal wire guide for introduction;
a multi-lumen body which provides for minimally invasive low profile surgical radial or femoral approach, integral wire guide access, along with three dimensional shape aspects defined by vessels being addressed, which is respectively lockable in place and one hand operable.

2. The improved steerable neurovascular catheter of claim 1, wherein the wire guide moves from a fully inserted to fully retracted position to cause a change, in the distal tip, from a first-elongated or straight configuration and to a second fully pre-formed position and shape, and a third pre-formed position and shape, along a plane ordinal to the multi-lumen body's axis.

3. The improved steerable neurovascular catheter of claim 2, wherein each respective positon may be locked, by means of a button manipulated radial to a plane ordinal to the multi-lumen body's axis.

4. The improved steerable neurovascular catheter of claim 3, each said respective insertion and retraction position marked by a button, actuable by one hand of a user.

5. The improved steerable neurovascular catheter of claim 4, having button actuated retraction and inserting functions, operable as a metered sliding button mechanism, manipulated in a linear fashion relative to a plane ordinal to the multi-lumen body's axis.

6. The improved steerable neurovascular catheter of claim 5, further comprising button actuated deflection, linearly along the metered sliding button pathway.

7. The improved steerable neurovascular catheter of claim 6, having a low-profile hub and port structure all actuated by one hand.

8. A system for neurovascular guidance, comprising, in combination:
at least one of a stainless steel, braided and Nitinol® types of additional medical devices;
a multi-lumen body modularly comprises parts for contrast about an over-the-wire low profile configuration; and,
at least about a 7 to 9 Fr dimensions.

9. The system of claim 8, further comprising PTE enhancers.

10. The system of claim 9, further comprising coils, with markers.

11. The system of claim 10, further comprising a customized ergonomic luer manifold with integral wire guide, which allows immediate one-handed ergonomic access without ever losing contact of the assembly and catheter during manipulation.

12. The system of claim 11, further comprising an integral wire guide with a deflatable distal tip.

13. The system of claim 12, further comprising variable and lockable tip deflection.

14. The system of claim 13, further comprising co-extruded catheter tubing having a predetermined three-dimensional configuration to facilitate steerable insertion and hand to govern complex catheter tip movements.

15. The system of claim 14, further comprising:
improved distal navigation, with improved proximal and distal stability, to prevent or mitigate air embolization by variable flexibility and stiffness distally.

16. The system of claim 15, further comprising extra soft molded tip with markers.

17. The system of claim 16, whereby a fully inserted wire guide results in a straight tip, capable of one-hand lockage and unlockable functionality achieved by lateral movement of the distal button.

18. The system of claim 17, wherein a wire guide fully retracted results in a hooked or curved orientation of the tip of the deflectable distal tip, and is lockable and un-locked with one hand of a user.

19. The system of claim 18, wherein when a guide catheter is in a locked position, it facilitates advancement of wires of microcatheters within the neurovascular guide catheter lumen, thus improving guide catheter stability and pushability of these wires and microcatheters.

20. The system of claim 19, wherein when a guide catheter is in an unlocked position, it favors the advancement of the guide catheter itself around sharp turns within the arterial or venous systems.
